(19) United States
(12) Patent Application Publication
(10) Pub. No.: US 2011/0213327 A1
(43) Pub. Date: Sep. 1, 2011

(54) Reusable insert for a diaper

(76) Inventors: Constantin Adrian Oprita, Jupiter, FL (US); Ioana Weber, Jupiter, FL (US)

(21) Appl. No.: 13/035,561

(22) Filed: Feb. 25, 2011

Related U.S. Application Data
(60) Provisional application No. 61/339,054, filed on Feb. 26, 2010.

(51) Int. Cl. A61F 13/505; D05B 23/00

(52) U.S. Cl. 604/385.14; 112/475.09

(57) ABSTRACT

A reusable insert for a diaper having a tubular shape with an absorbent material with an upper half and a lower half, and a sheet of water proof material sewn onto an outer side of the upper half so that the lower half of the absorbent material can be inserted inside the upper half to form twice as many layers of absorbent material with the water proof sheet on the outside surface.

### CROSS-REFERENCE TO RELATED APPLICATIONS

This application claims the benefit to a Provisional Patent Application 61/339,054 filed on Feb. 26, 2010 and entitled REUSABLE DIAPER WITH MULTI-LAYERED ABSORBENT ARTICLE.

### GOVERNMENT LICENSE RIGHTS

None.

### BACKGROUND OF THE INVENTION

1. Field of the Invention

The present invention relates generally to reusable absorbent articles such as reusable diapers, training pants, adult incontinence garments, feminine care pads that have the function of absorbing and containing body exudates.

Description of the Related Art including information disclosed under 37 CFR 1.97 and 1.98

Disposable absorbent articles such as disposable diapers, adult incontinence garments, feminine care pads and disposable training pants have the function of absorbing and containing body exudates. Once the absorbent core component of the above mentioned items is saturated with bodily discharges, such as urine, the entire absorbent article is usually discarded. Although some contemporary disposable diapers are made with biodegradable material, the greatest problem comes from the liquid impermeable material of the back sheet which is non-biodegradable and, consequently, inhibits the breakdown of the diaper, when thrown away. As a result of the environmental drawbacks associated with the disposable absorbent articles, reusable absorbent articles have become a popular alternative for consumers.

A reusable diaper generally includes an inner layer configured to wick moisture from a diaper wearer's body and an outer layer configured to be substantially liquid impervious. A reusable diaper can have a pocket defined as a space between the inner and outer layers where at least one liquid absorbent insert can be placed. In this case the diaper includes a slit in the inner layer that provides access into the space between the inner and outer layers. Such examples can be found in U.S. Pat. No. 7,629,501 issued to Labit at al. on Dec. 8, 2009 and entitled REUSABLE DIAPERS; and U.S. Pat. No. 6,579,273 issued to Dupuy on Jun. 17, 2003 and entitled REUSABLE BABY DIAPER HAVING REUSABLE ABSORBENT INSERT.

The disadvantage of the above-mentioned pocket diapers is that once the absorbent insert is saturated with the bodily discharges, the absorbent insert and the entire diaper must be washed. If only the insert is removed and replaced with a new one, the inner layer of the reusable diaper, placed against the body of the wearer, still contains the various exudates discharged from the body and is not completely sanitary. If the reusable diaper does not have a pocket and the absorbent article is placed inside the diaper, being in direct contact with the wearer's body and not covered by the inner layer of the reusable diaper, after the single absorbent insert is saturated with bodily discharges, it has to be removed and replaced with a new one.

### BRIEF SUMMARY OF THE INVENTION

An improved absorbent article, to be used in a reusable diaper, has one or a plurality of reusable absorbent inserts that can be independently removed without removal of the diaper from the wearer, thus extending the diaper's time of usage and reducing the frequency of changing the reusable diaper or over-garments.

In one embodiment, the insert is tubular in shape in which one end of the insert can be inserted into the inside of the other end to form a multiple layer surface for improved absorbency and ease in cleaning.

A process for making the insert in which a sheet of absorbent material having four section has a second sheet of a water proof material sewn into one of the four section, the absorbent sheet is then folded into half and sewn around the open sides to form a tube closed on both ends with the water proof material located on the outer surface of the tube. The half of the insert without the water proof material is then inserted into the inner sides of the half with the water proof material to form the finished insert.

### BRIEF DESCRIPTION OF THE SEVERAL VIEWS OF THE DRAWINGS

FIG. 1 is an outside view of a reusable diaper with an absorbent article embodiment comprised of three absorbent inserts and a fastening system operable to attach one or more absorbent inserts to each other.

FIG. 2 is an outside view of the absorbent article embodiment comprised of three absorbent inserts and a fastening system operable to attach one or more absorbent inserts to each other.

FIG. 3 is a cross section view of one of the reusable absorbent inserts included in the absorbent article embodiment showing a plurality of layers of suitable material configured to absorb and store liquids therein and one substantially liquid-impervious layer to thereby resist wicking of moisture.

FIGS. 4 is a plan view of the absorbent article embodiment shown in FIG. 2 where the dotted line is depicting an alternate shape of the liquid impervious layer included in one or all the absorbent inserts.

FIG. 5 is a plan view of the absorbent article embodiment shown in FIG. 2 where the dotted line is depicting an alternate shape of the liquid impervious layer included in one or all the absorbent inserts.

FIG. 6 shows an alternate embodiment of the absorbent article shown in FIG. 2 depicting an alternative shape of the comprising absorbent inserts, in the wing areas, and the fastening system operable to attach one or more absorbent inserts, to each other.

FIG. 7 shows an alternate embodiment of the absorbent article shown in FIG. 2 depicting an alternative shape of the comprising absorbent inserts, in the wing areas, and the fastening system operable to attach one or more absorbent inserts, to each other.

FIG. 8 shows a partial view of an alternate embodiment of the absorbent article shown in FIG. 2 depicting an alternate shape of the comprising absorbent inserts, in the wing areas, and the fastening system operable to attach one or more absorbent inserts, to each other.

FIG. 9 shows a partial view of an alternate embodiment of the absorbent article shown in FIG. 2 depicting an alternate shape of the comprising absorbent inserts in the wing areas and the fastening system operable to attach one or more absorbent inserts, to each other.

FIG. 10 shows a partial view of an alternate embodiment of the absorbent article shown in FIG. 2 depicting an alternate shape of the comprising absorbent inserts, in the wing areas, and the fastening system operable to attach one or more absorbent inserts, to each other.

FIG. 11 shows an alternate embodiment of the absorbent article shown in FIG. 2 depicting an alternate structure of the absorbent inserts, with elastic disposed in the leg cuffs area.

FIG. 12 shows an alternate embodiment of the absorbent article shown in FIG. 2 depicting different widths of the comprising absorbent inserts, in the leg cuff area.

FIG. 13 shows an alternate embodiment, of the absorbent article shown in FIG. 2 comprising an expandable absorbent insert and a second non-expandable absorbent insert located inside the pocket created in the expandable absorbent insert, when folded.

FIG. 14, FIG. 15, FIG. 16, FIG. 17, FIG. 18, FIG. 19, FIG. 20, FIG. 21 and FIG. 22 show the manufacturing process stages of an alternate embodiment of the absorbent article shown in FIG. 2.

FIG. 23 is depicting an embodiment of the expandable absorbent insert shown in FIG. 13 where the shape of the absorbent fabric used in manufacturing the expandable absorbent insert is not rectangular. It has angulated edges thus allowing an easier folding and unfolding process for the expandable insert.

FIG. 24 is showing the expandable absorbent insert shown in FIG. 13 in the folded position.

FIG. 25 is showing the expandable absorbent insert shown in FIG. 13 in the folded position with the non-expandable absorbent insert shown in FIG. 13 inside the pocket of the folded expandable absorbent insert.

FIG. 26 is depicting an embodiment of the absorbent article shown in FIG. 13 comprising the expandable absorbent inserts and the non-expandable absorbent insert where a fastening system, operable to attach the absorbent inserts to each other has been used, in the unfolded position.

FIG. 27 is depicting an embodiment of the absorbent article shown in FIG. 13 comprising the expandable absorbent inserts and the non-expandable absorbent insert where a fastening system, operable to attach the absorbent inserts to each other has been used, in the folded position.

FIG. 28 is depicting an alternate embodiment of the absorbent article 20, shown in FIG. 2 comprising a pocket style absorbent insert and a non-expandable absorbent insert.

### DETAILED DESCRIPTION OF THE INVENTION

A reusable, waste-containment diaper with an improved absorbent article is enclosed. According to various aspects, exemplary embodiments are provided of improved absorbent articles.

In one exemplary embodiment, a reusable diaper generally includes an inner layer and an outer layer configured to be substantially liquid-impervious. The inner layer, in this case, can be soft fabric that inhibits contact between the diaper wearer and the outer layer, in the areas around the absorbent article, for comfort. The absorbent article is located in the crotch portion of the diaper and is extending toward the rearward and forward portions of the diaper. Elastic strips can be attached to each side of the crotch portion in the leg cuff areas. The reusable diaper can be contoured in the classic hour glass shape when open, having elongated wings along the rearward portion which overlap the forward portion when the diaper is folded in half, thereby forming waist closure and leg openings. The reusable diaper can be fitted with snap closures arranged to provide waist adjustment. Different connector members, besides snaps, can also be used.

A reusable, waste-containment absorbent article is also disclosed for use inside an over-garment such as a conventional reusable diaper, or a disposable diaper, or over-pants, or the combination of a reusable diaper covered with over-pants. The absorbent article comprises one or a plurality of reusable absorbent inserts, a fastening system operable to attach or detach one or more absorbent inserts.

An absorbent article is a combination of one or more absorbent inserts, suitable for absorbing, distributing, and storing aqueous fluids such as body exudates. Each absorbent insert can contain one or more layers of substantially liquid impervious material to thereby resist wicking of moisture and the transfer of moisture from one absorbent insert to the other. An absorbent article absorbs and contains body exudates by use of absorbent inserts, and, more specifically, refers to devices which are placed against or in proximity to the body of the wearer to absorb and contain the various exudates discharged from the body.

It should be understood, however, that the present invention is applicable to absorbent articles to be used in reusable diapers or any other similar garments, such as incontinence briefs, incontinence under-garments, diaper holders and liners, training pants, pull-on diapers, and the like.

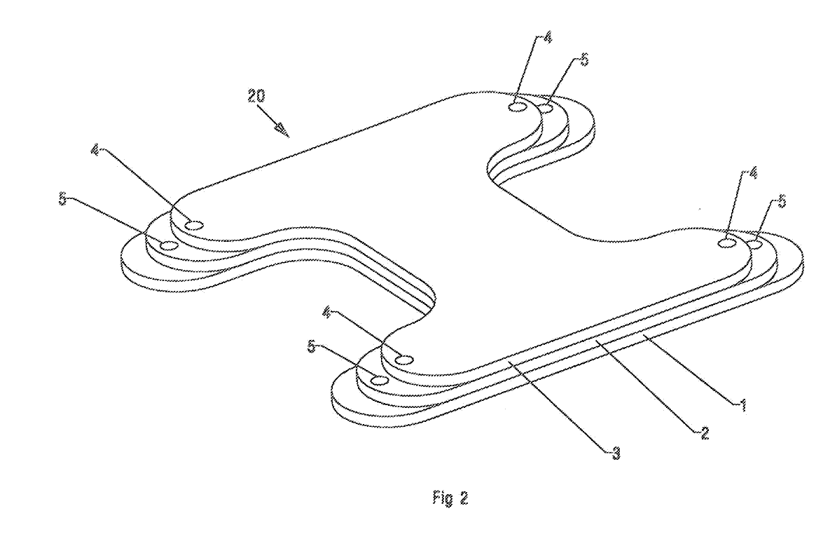

One embodiment of the present invention is the disposable absorbent article 20 as shown in FIG. 2. The absorbent article 20 from the present invention can be placed in reusable or disposable diapers, incontinence briefs, incontinence under-garments, diaper covers, diaper liners, training pants, pull-on diapers, an the like.

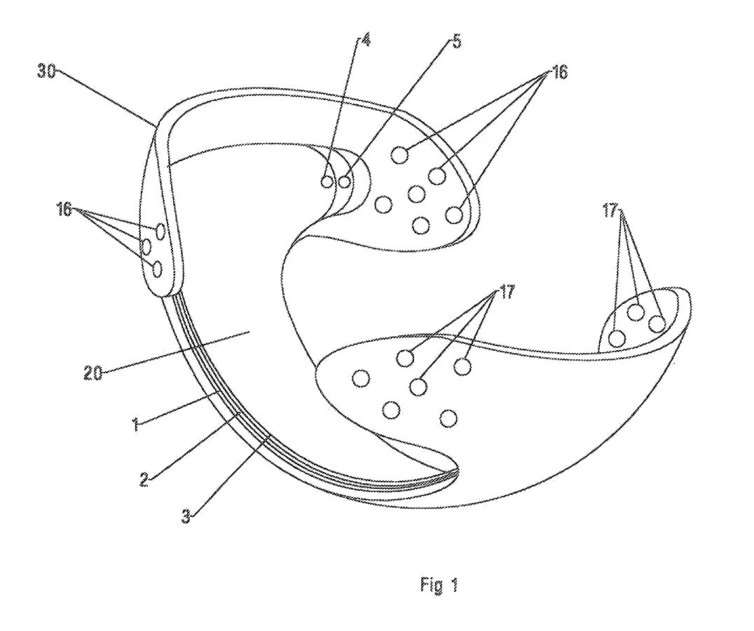

FIG. 1 shows in perspective a reusable diaper 30 with an absorbent article 20 secured on an inner surface, the absorbent article 20 having three absorbent inserts 1, 2 and 3 and a fastening system 4 and 5 operable to attach one or more absorbent inserts to each other to form layers of individually removable inserts. The reusable diaper 30 is fitted with snaps 16 and 17 or hook-and-loop closures, combination thereof, adhesives, buttons, clasps, Velcro, combination thereof, or any suitable fastening system, arranged to provide waist adjustment when the elongated wings along the rearward portion overlap the forward portion, thereby forming waist closure and leg openings.

FIG. 2 shows in perspective view of an absorbent article 20, according to the present invention, that contains absorbent inserts 1, 2, 3 and a fastening system 4, 5 operable to removably attach one or more inserts to each other. The multi-piece absorbent article 20 comprising multiple absorbent inserts such as absorbent inserts (1, 2, 3) is more fully illustrated and described below. By forming the absorbent article 20 with multiple components, several results are obtained. The improved absorbent article has a plurality of reusable absorbent inserts (1, 2, 3) that can be independently removed, without removal of the diaper from the wearer, thus the absorbent article usage time is increased and the frequency of changing the reusable diaper is reduced. Once the absorbent insert closest to the wearer's skin is saturated with bodily discharges, such as urine, the saturated absorbent insert 1 is removed, the next clean insert 2 will become the closest to the wearer's skin, the suction of the absorbent article is regenerated, and once again the article becomes capable of absorbing moisture. Thus, the layers of absorbent inserts 1, 2, 3 can be removed one at a time until all the inserts 1, 2, 3 are saturated. Upon removal of a saturated insert, the next insert acts to absorb any additional fluids. Therefore, the reusable diaper may be worn longer and regeneration of the absorbent article may be made without removal of the diaper from the wearer.

A second advantage is the versatility of this improved absorbent article which can be used in a multitude of garments such as reusable or disposable diapers, incontinence briefs, incontinence under-garments, diaper covers, diaper liners, training pants, pull-on diapers. A third benefit resulting from this improved design is the leakage reduction. Because each of the absorbent inserts (1, 2, 3) can have a layer configured to be substantially liquid-impervious, there is an increased leakage safety barrier between the multiple layers of absorbent inserts (1, 2, 3) or between the absorbent article 20 and the wearer's reusable or disposable diapers, incontinence briefs, incontinence under-garments, diaper covers, diaper liners, training pants, pull-on diapers, an the like. When one absorbent insert is saturated with bodily discharges and removed, the other layers are still dry and sanitary.

FIG. 3 is a cross section view of one of the layers of the inserts (1, 2, 3) included in the absorbent article embodiment 20 from FIG. 2, showing a plurality of permanently attached layers 8, 9, 10 of suitable material, configured to absorb and store liquids therein and one layer 7 of substantially liquid-impervious material to thereby resist wicking of moisture between the absorbent inserts. The absorbent insert 3 may comprise one or more permanently attached layers 8, 9, 10 of microfibers, hemp, hydrocolloid materials or any other suitable material configured to absorb and store liquids therein and also one layer 7 configured to be substantially liquid-impervious. Preferably, layer 7, configured to be substantially liquid-impervious, should be the closest to the next absorbent insert 2, from FIG. 2 to thereby resist wicking of moisture between inserts. Stitching or any other attachment method can be used for permanently connecting layers 7, 8, 9 and 10 between each other and forming the unitary absorbent insert.

The absorbent insert 3, included in the absorbent article embodiment 20, is attachable to the absorbent insert 2 with a fastening system 4 which can be snaps, hook-and-loop closures, combination thereof, adhesives, buttons, clasps, Velcro, combination thereof, or any suitable fastening system.

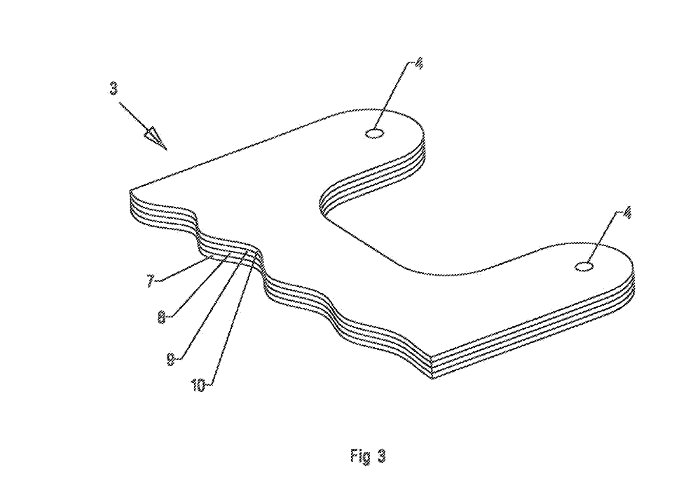

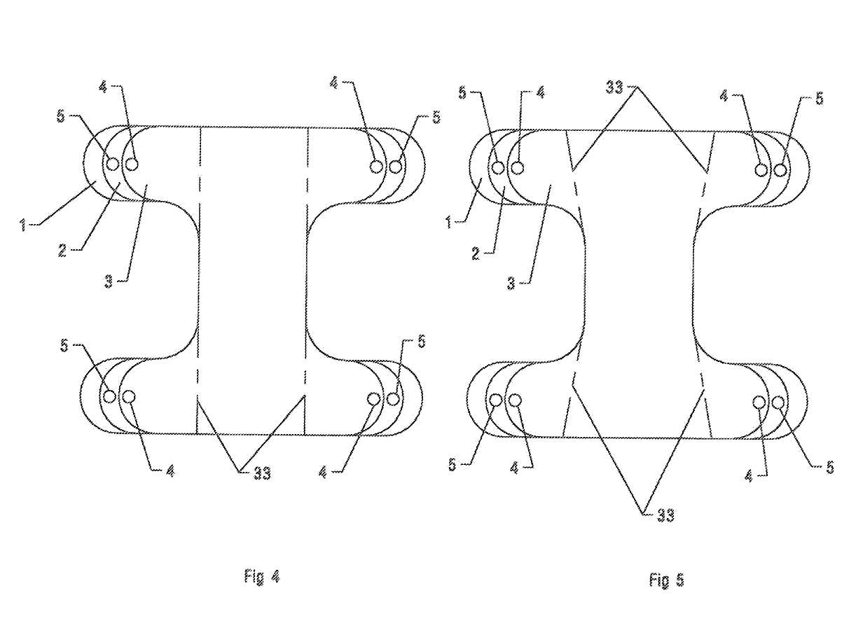

FIG. 4 and FIG. 5 are plan views of embodiments of the absorbent article 20 shown in FIG. 2 where the dotted line 33 is depicting an alternate shape of the liquid-impervious layer 7, shown in FIG. 3, included in one or all of the absorbent inserts. In some embodiments, the liquid impervious layer 7 can be smaller than the rest of the absorbent insert or it can have the same shape as the rest of the layers.

FIG. 6 shows an alternate embodiment of the absorbent article 20, shown in FIG. 2, depicting an alternative shape of the absorbent inserts 1, 2 and 3. Multiple releasable fastening devices such as snaps 4, 5, 18 and 19 are used in this embodiment.

FIG. 7 shows another embodiment of the absorbent article 20, shown in FIG. 2, depicting an alternate shape of the absorbent inserts 1, 2 and 3 and the releasable fastening system 4 and 5 operable to attach one or more absorbent inserts, to each other. In this case, the absorbent inserts 1, 2 and 3 have the same width, on the entire length. The FIG. 7 embodiment does not include the four outward extending wings as shown in FIG. 6. The FIG. 7 embodiment is a rectangular shaped insert.

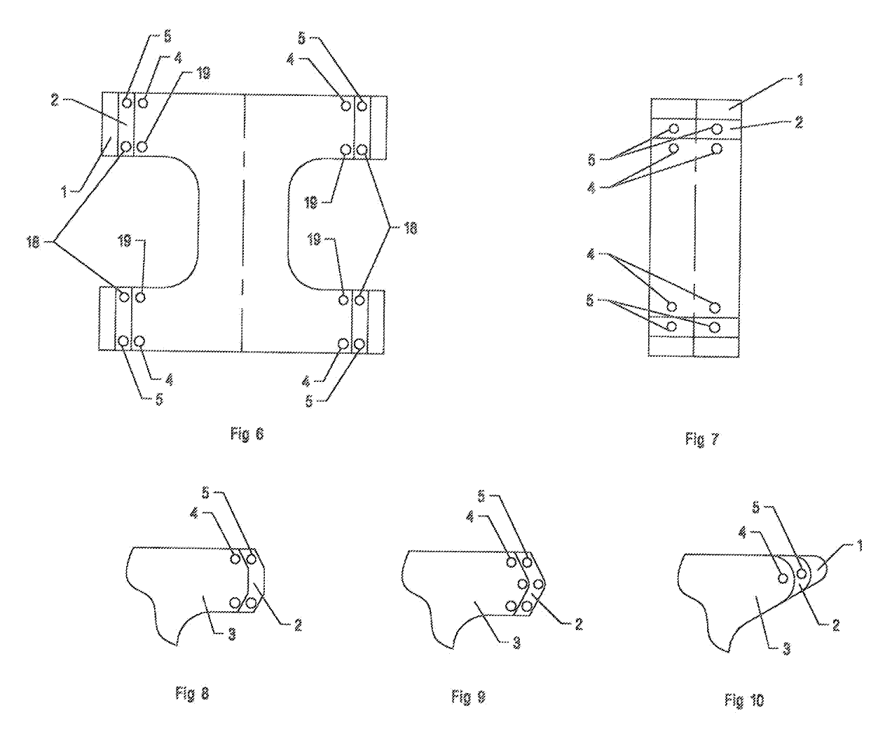

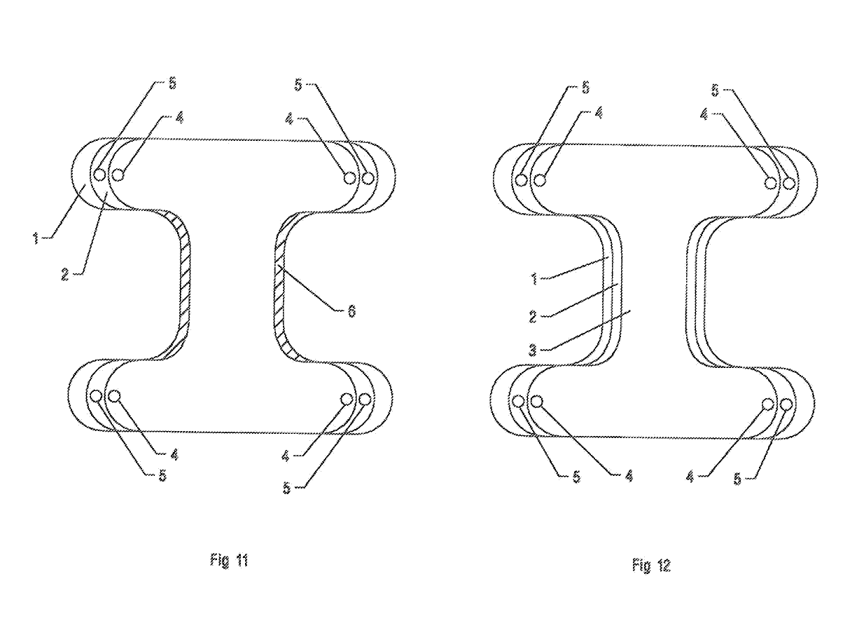

FIGS. 8, 9 and 10 show partial views of an alternate embodiment of the absorbent article 20, shown in FIG. 2, depicting alternate shapes of the areas containing the fastening system 4 and 5 (for example, snaps) operable to attach one or more inserts to each other.

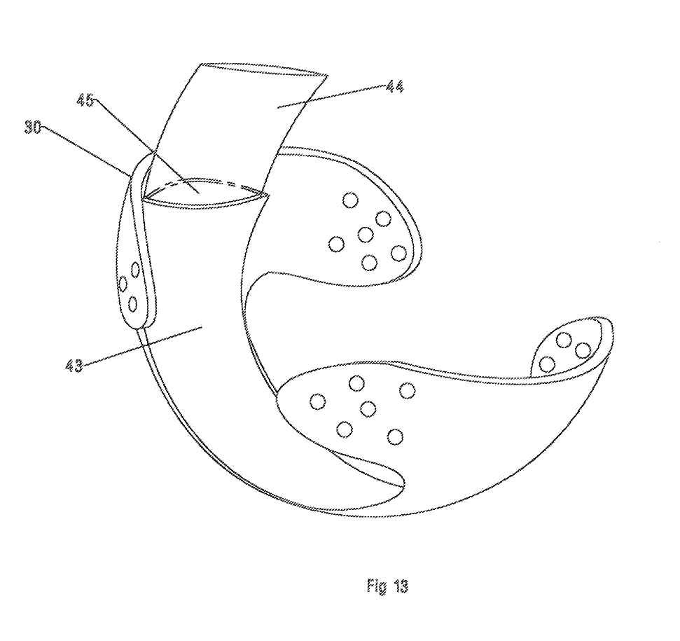

FIG. 11 shows an alternate embodiment of the absorbent article 20, shown in FIG. 2, depicting an alternate structure of the absorbent inserts (1, 2, 3) with an elastic material 6 disposed in the cuffs area. One or more absorbent inserts can have elasticized edges 6 in the crotch portion 12 thus creating an additional leakage barrier in the leg cuffs area.

FIG. 12 shows an alternate embodiment of the absorbent article 20, shown in FIG. 2, depicting different widths of the comprising absorbent inserts (1, 2, 3) in the leg cuff area.

FIG. 13 shows an alternate embodiment of the absorbent article 20 shown in FIG. 2 that includes an expandable absorbent insert 43 and the non-expandable absorbent insert 44 that is inserted into the expandable insert with both expandable and non-expandable inserts placed inside the reusable diaper 30. The expandable insert 43 is described more below.

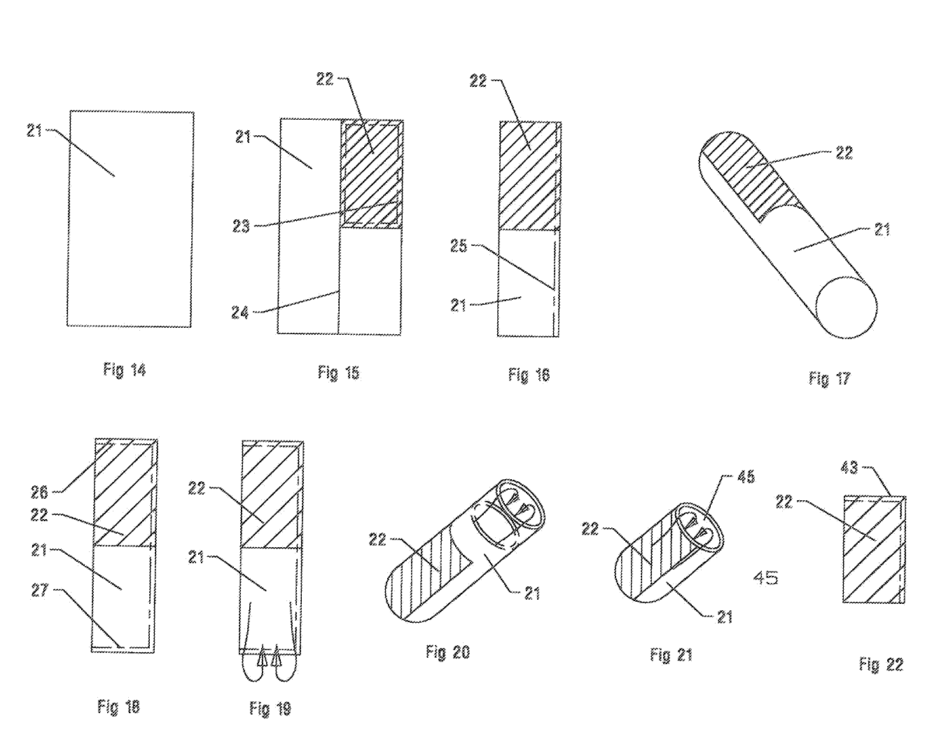

FIGS. 14 through 22 show the process steps used to form the expandable absorbent insert 43 shown in FIG. 13. The expandable absorbent insert 43 has the advantage of allowing faster drying time after being laundered because it is unfolding (opening up) in the laundering process. At the same time it offers increased absorbency due to the number of layers of absorbent fabric that result from folding it, as shown in FIGS. 19 through 22. In addition to the layers of absorbent fabric, it contains one substantially liquid-impervious layer 22 to thereby resist wicking of moisture.

The process to form the expandable absorbent insert 43 includes starting with a sheet of absorbent material 21 that is to absorb and store bodily fluids. The insert 43 has four sections with a sheet of water proof material 22 being sewn to one of the four sections as seen in FIG. 15. The insert 43 is then folded over as seen in FIG. 16, along the folding line 24 as seen in FIG. 15, with the water proof material 22 on the outside. The longest open side of the insert 43 is then sewn to form a tube with open ends as seen in FIG. 17. The two remaining open ends are then sewn together as seen in FIG. 18 to form a tubular shaped insert 43 with the water proof material 22 on the outside of one of the ends as seen in FIG. 18. With this construction, the tubular insert 43 can then have the one end without the water proof material 22 inserted inside the other end as seen in FIGS. 19, 20 and 21 to form the completed expandable absorbent insert 43 as seen in FIG. 21 and to form an insert with twice as many layers as the one that is not folded inside. FIG. 22 shows the folded insert 43 in a flattened position that is ready to be placed inside a diaper such as a cotton reusable diaper.

FIG. 14 is showing one sheet of material 21 configured to absorb and store liquids.

FIG. 15 is showing the same sheet of absorbent material 21 on which a layer of liquid impervious material 22 has been fastened, through sewing or similar methods. The crosshatched area in this figure is representing the liquid-impervious material layer 22 and the dotted line 23 is the stitching perimeter for fastening layers 21 and 22 to each other.

FIG. 16 is showing the absorbent material layer 21 with the liquid-impervious fabric layer 22 permanently attached to it, folded in two, along line 24 shown in FIG. 15, with layer 22 being on the outside. A new stitching contour is represented by the dotted line 25 which has the purpose of creating a tube out of the layers of fabric 21 and 22, which tube is open at both ends.

FIG. 17 is showing an outside view of the fabric layers 21 and 22, in the shape of the tube, open at both ends.

FIG. 18 is an outside view of the fabric layers 21 and 22, permanently connected to each other, in a flat position. Stitching contours 26 and 27 are closing the tube at both ends.

FIG. 19 is showing the direction of pushing the lower half of the tube inside the upper half of the tube.

FIGS. 20 and 21 are showing the progress of the lower half of the tube, while being pushed inside the upper half.

FIG. 22 is a plan view of the expandable absorbent insert where four layers of suitable material configured to absorb and store liquids therein have been created through the process of pushing one end of the tube into itself. The layer of liquid impervious material 22 will remain on the outside of the absorbent insert. The process of pushing one end of the tube into itself has created a pocket inside the expandable absorbent insert 43, shown in FIG. 13.

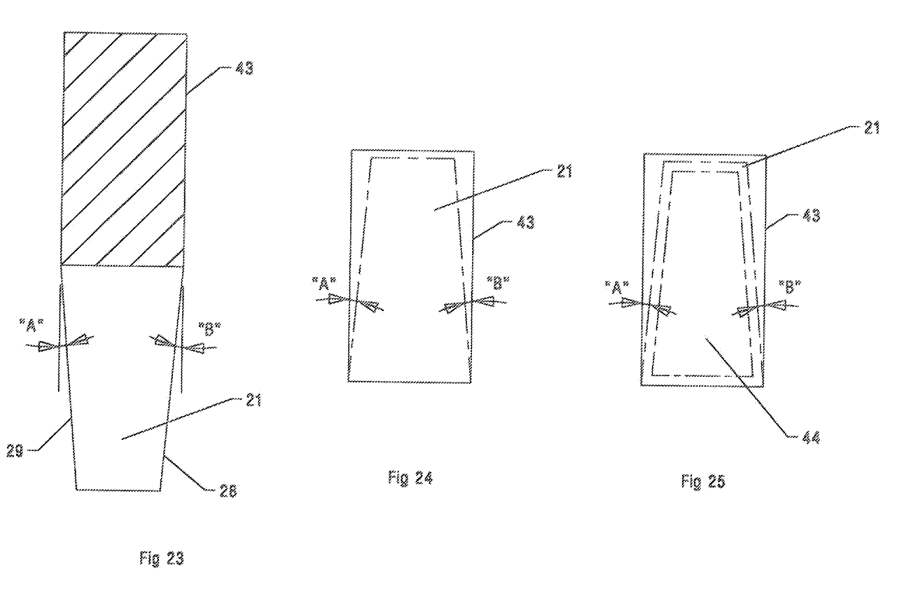

FIG. 23 shows an embodiment of the expandable absorbent insert 43 shown in FIG. 13 in which the insertable end is tapered for easy insertion into the other end. The shape of the absorbent fabric layer 21 used in manufacturing the expandable absorbent insert 43 is not rectangular. It has angulated edges 28 and 29. By creating the edges 28 and 29 at an angle, “A” and “B”, the process of pushing the lower half of the tube into the upper half is easier. In addition, this angled structure allows the unfolding of the insert, by itself, while being laundered thus avoiding the contact with the various exudates discharged from the body, if the unfolding of the expandable absorbent insert is needed, prior to the laundering process. FIG. 24 is showing the expandable absorbent insert 43 in the folded position.

FIG. 25 is showing the expandable absorbent insert 43 of FIG. 24 in the folded position with the non-expandable absorbent insert 44 inside the pocket of the folded expandable absorbent insert 43. This adds additional layers of absorbent material to the absorbent article. The process of pushing one end of the tube into the other end creates a pocket 45 as shown in FIGS. 13 and 21 inside the expandable absorbent insert 43.

FIG. 26 shows an embodiment of the absorbent article comprising the expandable absorbent insert 43 and the non-expandable absorbent insert 44 where a fastening system 88 is used to attach the second absorbent insert 44 to the first absorbent insert. The non-expandable absorbent insert is basically a flat sheet of material as opposed to the expandable insert 43 that is sewn on the three sides to form a tubular shaped insert capable of having one end inserted inside the other end. The advantage of this embodiment is that, if needed, prior to laundering, the expandable absorbent article can be manually unfolded by pulling on the outer edge 78 of the non-expandable absorbent insert 44. When the non-expandable absorbent insert 44 is pulled out of the pocket 45 created in the expandable absorbent insert 43 when folded, it will also unfold the expandable absorbent insert 43 because both inserts are connected by the fastening system 88.

FIG. 27 is showing the expandable absorbent insert 43 in the folded position with the absorbent insert 44 inside the pocket 45 of the expandable absorbent insert 43 and the fastening system 88 operable to attach the absorbent inserts to each other. The fastening system 88 includes one or more snaps, hook-and-loop closures, combination thereof, adhesives, buttons, clasps, Velcro, combination thereof, or any suitable fastening system. It should be understood, however, that the present invention is applicable to one or a plurality of absorbent inserts that can be placed inside the pocket 45, created inside the folded expandable absorbent insert 43, thus allowing an increase or decrease of the absorbency level of the absorbent article.

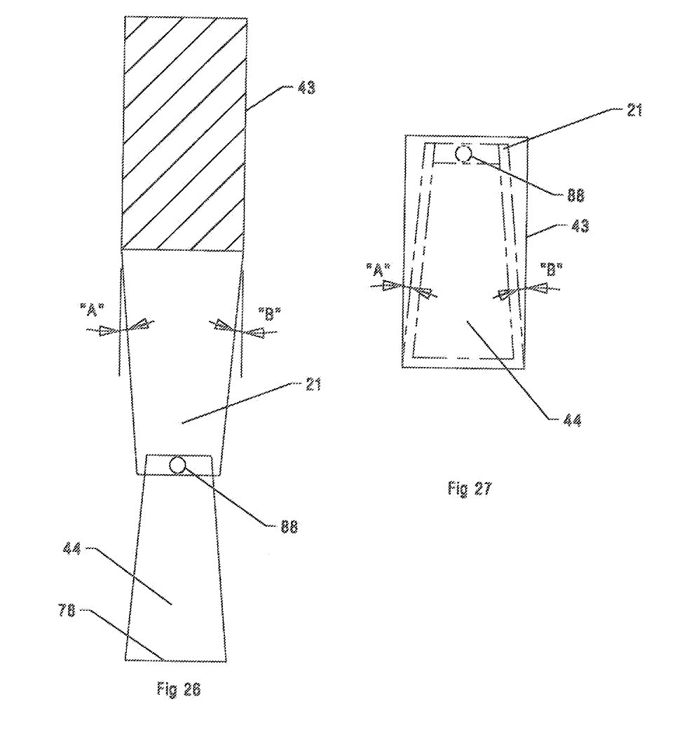

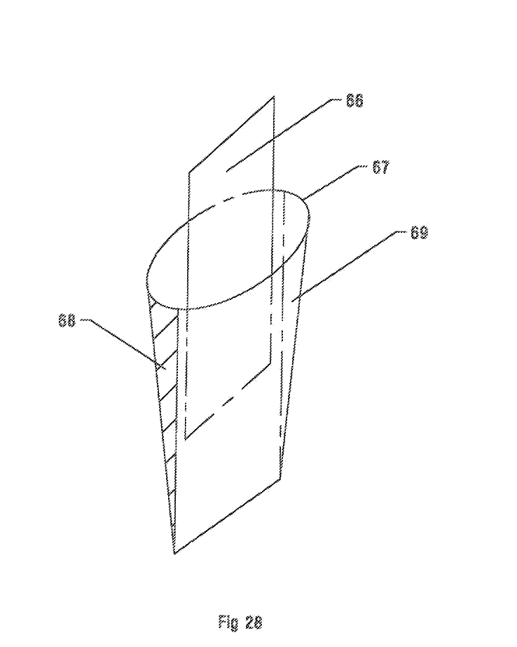

FIG. 28 shows another embodiment of the expandable absorbent insert with a pocket shaped piece 67 open on one end for insertion of a sheet of absorbent material 66. The pocket 67 is formed from an absorbent material 69 with a water proof material 68 sewn onto one of the outer sides. One or more sheets of the inner absorbent material 66 can be inserted into the pocket 67 or one thicker sheet of absorbent material 66 can be used in order to increase the overall absorbency of the absorbent article that is then placed into an inner side of a diaper.

The advantage of the absorbent insert shown in FIGS. 13 through 28 is that it will dry much faster than a thicker sheet of material that has the same overall thickness, thus saving time and energy. The absorbent insert is easily shaped by inserting one end into the other end to form additional layers of absorbent material for use in a diaper such as a cotton reusable diaper. Both the reusable diaper and the absorbent insert 43 of the present invention can be made out of green, eco-friendly materials such as bamboo and do not need to be disposed of like the prior art plastic diapers. The insert 43 of the present invention can be easily removed from the diaper and placed in a washing machine where it will unfold itself in the cleaning process. The absorbency of the insert 43 can be increased by adding additional sheets of the absorbent material to the inside of the expandable insert. Thus, any amount of absorbency can be established.

We claim the following:

1. A reusable insert for a diaper comprising:
a tubular shaped piece made from an absorbent material being closed at both ends and forming an upper half and a lower half;
a piece of water proof material sewn onto an outer surface of one half of the upper end of the tubular shaped absorbent piece; and,
the lower half of the tubular shaped piece being capable of inserting into an inner surface of the upper half with the water proof piece on the outer surface.

2. The reusable insert of claim 1, and further comprising:
the lower half of the absorbent material is tapered toward an outer end.

3. The insert of claim 1, and further comprising:
an additional sheet of absorbent material is placed within the inside of the lower half when the lower half is placed within the upper half.

4. The reusable insert of claim 3, and further comprising:
the additional sheet of absorbent material is removably secured to the lower half of the absorbent material.

5. The reusable insert of claim 4, and further comprising:
the lower half of the absorbent material and the additional sheet of absorbent material are both tapered in a direction towards one another.

6. An insert for a diaper comprising:
a pocket piece having three sides closed and a fourth side open;
the pocket piece being formed from an absorbent material;
a water proof material sewn onto an outer surface of one half of the pocket piece; and,
a sheet of an absorbent material inserted inside the pocket.

7. A process of forming a reusable insert for use in a diaper, the process comprising the steps of:
forming a first sheet of absorbent material having four corners;
sewing a second sheet of water proof material to one of the four corners of the first sheet of absorbent material;
folding over the first sheet of absorbent material with the water proof sheet on an outer side;
sewing the three open sides of the absorbent material together to form a tubular shaped piece with two closed ends; and,
inserting the one end of the absorbent material without the water proof sheet inside of the other end of the absorbent material with the resulting insert having doubled layers adjacent to the waterproof material.
